(19) United States
(12) Patent Application Publication
(10) Pub. No.: US 2010/0004196 A1
(43) Pub. Date: Jan. 7, 2010

(54) HEPARAN SULFATE PROTEOGLYCAN COMPOSITION AND USE THEREOF

(75) Inventors: Ariane De Agostini, Genthod (CH); Robert J. Linhardt, Albany, NY (US)

Correspondence Address: BAKER & DANIELS LLP, 300 NORTH MERIDIAN STREET, SUITE 2700, INDIANAPOLIS, IN, 46204, US

(73) Assignees: Hopitaux Universitaires De Geneve, Geneva (CH); Rensselaer Polytechnic Institute, Troy, NY (US)

(21) Appl. No.: 12/486,272

(22) Filed: Jun. 17, 2009

Related U.S. Application Data
(60) Provisional application No. 61/132,241, filed on Jun. 17, 2008.

(51) Int. Cl. A61K 31/726; C07H 5/06; C12Q 1/02; C12M 1/00; A61P 7/00; A61P 35/00; A61P 29/00; A61P 15/08

(52) U.S. Cl. 514/54; 536/55.3; 435/29; 435/287.1

(57) ABSTRACT

The present invention relates to method for the preparation of glycosaminoglycan compositions, isolated glycosaminoglycan compositions obtainable therefrom, glycosaminoglycan compositions, kits and use thereof. More specifically, the present invention provides a method for isolating glycosaminoglycan compositions of the invention from human follicular fluid. The compositions, related methods and uses according to the present invention are useful in the treatment and/or prevention of thrombotic diseases, cell proliferation disorders, proteolysis and inflammation mediated cell invasion and infertility.

This application claims priority from U.S. Provisional application 61/132,241 filed Jun. 17, 2008, the subject matter of which is incorporated herein by reference.

### FIELD OF THE INVENTION

The present invention relates to methods for the preparation of glycosaminoglycan compositions, isolated glycosaminoglycan compositions obtainable therefrom, glycosaminoglycan compositions, kits and use thereof. More specifically, the present invention provides a method for isolating glycosaminoglycan compositions from human follicular fluid. The compositions, related methods and uses according to the present invention are useful in the treatment and/or prevention of thrombotic diseases, cell proliferation disorders, proteolysis and inflammation mediated cell invasion. Further, compositions, related methods and uses according to the present invention are useful in the treatment of infertility and more specifically in the detection of follicular rupture at ovulation.

### BACKGROUND OF THE INVENTION

Heparan sulfate proteoglycans (HSPGs) are ubiquitously distributed on the surface of animal cells and are secreted into the extracellular environment. They have numerous important biological activities mediated through interactions with diverse proteins. HSPGs are composed of a core protein with covalently attached heparan sulfate (HS) chains formed by repetitive sulfated disaccharides subunits (uronic acid-(1→4)-D-glucosamine). The different length and variable sequence of sulfated disaccharides (variable substitution patterns of the disaccharide subunits with N-sulfate, O-sulfate and N-acetyl groups) generate the structural diversity required to form specific oligosaccharide binding sites for proteins such as growth factors, protease inhibitors or cell adhesion molecules.

Heparan sulfate is a member of the glycosaminoglycan (GAG) family of carbohydrates and, as heparin, another member of the GAG family, is constructed from the two monosaccharide building blocks, (i) uronic acid (which may be selected from α-L-iduronic acid (IdoA) and β-D-glucuronic acid (GlcA)); and (ii) β-D-glucosamine (GlcN) (which may be selected from N-sulfated (GlcNS) or N-acetylated (GlcNAc). The above uronic acids may be both 2-O-sulfated (Ido(2S) and GlcAc(2S)) and the β-D-glucosamines above may be both 6-O-sulfated (GlcNS(6S) and GlcNAc(6S)). Finally, the above GlcNS and GlcNS(6S) may also be 3-O-sulfated (GlcNS(3S) and GlcNS(3,6S)). Therefore, 24 uronic acid-(1→4)-D-glucosamine disaccharide combinations may be made from those monosaccharide building blocks.

However, HS primary structure differs significantly from that of heparin: the main disaccharide subunit present in HS structure is the unsulfated GlcA-GlcNAc disaccharide sequence (50% of the total disaccharide units), whereas, the heparin structure, contains mainly the disaccharide subunit IdoA(2S)-GlcNS(6S) (70-90%) (Rabenstein et al., 2002, Nat. Prod. Rep., 19, 312-331).

Heparin is a known and clinically used anticoagulant which binds to the enzyme inhibitor antithrombin III (AT-III), resulting in the activation of AT-III which then inactivates thrombin and other proteases involved in the blood clotting cascade, notably factor Xa.

AT-III binds to a specific pentasaccharide sulfation sequence contained within the heparin polymer: GlcNAc/NS(6S)-GlcA-GlcNS(3S,6S)-IdoA(2S)-GlcNS(6S), bearing a 3-O-sulfated glucosamine essential for Antithrombin (AT) binding (Rosenberg et al., 1997, J. Clin. Invest. 99, 2062-2070). Anticoagulant heparan sulfate (aHS) also share this AT-binding pentasaccharide motif.

The anticoagulant heparan sulfate proteoglycans (aHSPGs) are produced by endothelial cells and are thought to endow the vascular wall with antithrombotic properties but they are also abundant in the reproductive tract (De Agostini, 2006, Swiss Med Wkly, 136:583-590). Major tissue remodeling occurs in hormone-responsive tissues of the female genital tract, at ovulation and during gestation, involving proteolysis, fibrin deposition and tightly controlled inflammation. The expression of aHSPG in extra-vascular compartments of the female genital tract and the reproductive defects of knockout mice deficient in aHSPG outline the emerging role of aHSPG in the control of tissue remodeling in reproduction (De Agostini, 2006, above; HajMohammadi et al., 2003, The Journal Of Clinical Investigation, 11(7), 989-999). In the ovary, aHSPGs are strongly expressed in granulosa cells of pre-ovulatory follicles where they are co-localized with serine protease inhibitors involved in the control of proteolytic activities at ovulation. In extra-vascular compartments, aHSPGs are thought to contribute to the control of proteolysis and inflammation during tissue remodeling (De Agostini, 2006, above).

Thrombosis risk due to lipid-mediated endothelial activation is increasing with age in men and in post-menopausal women. Cycling women have decreased incidence of arterial thrombotic disease such as myocardial infarction, arterial thrombosis and ictus. At menopause, when the ovulatory cycle stops, the relative risk of these diseases increases to the level found in men of corresponding age. These factors involve atherosclerosis-related diseases related to inflammation-mediated endothelial activation, as well as to metabolically defined lipid profiles in plasma. The administration of hormones to reduce menopause-related health problems including cardiovascular disease risk remains highly controversial and has clearly been demonstrated to be both inefficient for cardioprotection and dangerous for breast and endometrial cancer risk. Therefore, due to the increase in life expectancy and aging of the population, prevention and/or treatment of thrombotic diseases has emerged.

Further, during angiogenesis, tumor cell invasion and blastocyte tissue invasion during implantation, the tissue penetration is possible through tissue remodeling implying a degradation of the extracellular matrix (ECM breakdown) by proteolytic enzymes (plasmin, metalloproteases) and by glycosidases (heparanase). Tissue remodeling is initiated by an acute local inflammation mediated by pro-inflammatory cytokines and chemokines that induce vascular permeabilization, and oedema. This inflammation is limited in time and space to allow destabilization of the existing tissue rendering it amenable to remodeling. Such reaction is well known at ovulation, when it occurs in response to a LH discharge. Cell adhesion molecules have emerged as being essential for the development of atherosclerosis and restenosis after angioplasty. In particular, the selectin family (L, E, and P) plays a crucial role for the earliest events in the inflammatory response, leading to the “rolling” phenomenon of the leukocytes on the vascular endothelium, followed by leukocyte migration which consists in the first step for polynuclear neutrophil recruitment during endothelial activation. P- and L-selectins also have a pathological role in diseases involving inflammation and reperfusion, as well as in carcinoma metastasis.

Further, ovulation disorders are a frequent cause of female infertility that often remains unexplained and the control of ovulation is a major concern in infertility treatments. The current means available to assess ovulation are ultrasound observation of changes in echographic image denoting the transition between preovulatory follicle and corpus luteum, hormonal measurements of the gonadotrophin LH and the gonadal steroid progesterone, but no direct observation of the follicular rupture is available. However, during fertility/infertility checks and/or infertility treatments on women, monitoring of the follicular rupture at ovulation would be really desirable.

Heparin is used to improve in-vitro fertilization (IVF) results and prevent recurrent spontaneaous abortions, despite the lack of understanding of the underlying mechanisms (Fiedler et al, 2004, Eur. J. Med. Res., 9:207-214). However, heparin use is limited by its side effects such as bleeding. Moreover, recent problems were encountered by the presence of contaminants (such as oversulfated chondroitin sulfate) in heparin derived from a mucous obtained from pig intestines and other animal tissues in China, which induced extreme allergic reactions.

There is therefore a need for new treatments for managing thrombosis risks in men or post-menopausal women or for treating thrombosis. There is a need as well for accurately monitoring ovulation in women undergoing fertility/infertility checks and/or infertility treatments and improving embryo implantation.

### SUMMARY OF THE INVENTION

The present invention relates to methods for the preparation of glycosaminoglycan compositions, isolated compositions obtainable therefrom, glycosaminoglycan compositions, kits and use thereof. The compositions, related methods and uses according to the present invention are useful in the treatment and/or prevention of thrombotic diseases, cell proliferation disorders, proteolysis, inflammation mediated cell invasion and infertility.

A first aspect of the invention provides a method for the preparation of a glycosaminoglycan composition.

A second aspect of the invention relates to an isolated glycosaminoglycan composition obtainable by a method according to the invention.

A third aspect of the invention relates to an isolated glycosaminoglycan composition comprising: (a) at least 2% 3-O-sulfated glucosamines; and (b) about 0.5 to 2 sulfate per disaccharide unit.

A fourth aspect according to the invention resides in a pharmaceutical preparation comprising at least one glycosaminoglycan composition according to the invention and pharmaceutically acceptable carrier or excipient.

A fifth aspect according to the invention relates to a use of a glycosaminoglycan composition according to the invention for the preparation of a medicament for the prevention and/or treatment of a disease selected from a thrombotic disease, a cell proliferation disorder such as a proliferative disease e.g. cancer, proteolysis and inflammation mediated tissue remodeling and cell invasion, and infertility.

A sixth aspect according to the invention relates to a glycosaminoglycan composition according to the invention for preventing and/or treating a disease selected from a thrombotic disease, a cell proliferation disorder such as a proliferative disease e.g. cancer, proteolysis and inflammation mediated tissue remodeling and cell invasion, and infertility.

A seventh aspect according to the invention relates in a method for preventing and/or treating a disease comprising the administration of a therapeutically effective amount of a glycosaminoglycan composition or a pharmaceutical composition according to the invention in a mammal in need thereof and wherein the disease is selected from a thrombotic disease, a cell proliferation disorder such as a proliferative disease e.g. cancer, proteolysis and inflammation mediated tissue remodeling and cell invasion, and infertility.

An eighth aspect of the invention resides in a method of detecting of follicular rupture at ovulation.

A ninth aspect according to the invention resides in a kit for determining the follicular rupture at ovulation of a female.

### DESCRIPTION OF THE FIGURES

FIG. 1 shows the fractionation of hFF aHSPG by MonoQ chromatography on a DEAE-Sephacel by a NaCl gradient (−) where the detected species are represented versus the elution volume (ml). Proteins are detected by OD₂₈₀ₙₘ (-•-), the aHSPG fraction by ¹²⁵I-AT ligand-binding assay (--) and the GAGs by Alcian Blue (-Δ-) as described in Example 1. The fractions pooled for further purification are indicated by a bar.

FIG. 2 shows the isolation of high Mᵣ aHSPG fraction from free heparan sulfate GAGs by gel filtration on Sepharose CL4B, where the detected species are represented versus the elution volume (ml). Proteins are detected by OD₂₈₀ₙₘ (-•-), the total HSPG is followed by a by Alcian Blue (-Δ-) and contains all aHSPG fraction (detected by ¹²⁵I-AT ligand-binding assay (--)) as described in Example 1. Elution position of heparin is indicated by an arrow. Fractions eluting pooled for further analysis are indicated by a bar.

FIG. 3 shows the ¹H-NMR spectra of standard HS, hFF derived aHS and iHS measured as described in Example 2. A: commercial standard HS; B: hFF aHS obtained as described in Example 1; C: hFF iHS obtained as described in Example 1. D: Expansion of spectra B and C between 4 and 5 ppm and the difference spectrum of B-C. a: H-1 GlcNAc; b: H-1 IdoA2S; c: H-1 IdoA; d: H-5 IdoA2S; e: H-1 GlcA; f: H-2 IdoA2S; g: H-3 IdoA2S; h: H-6 GlcNS6S or GlcNAc6S; i: H-4,5,6 GlcNAc; j: H-2 GlcNS. Panel D shows e′ present in B and the B-C difference spectrum corresponds to H-3 GlcNS3S and H-3 GlcNS3S6S.

FIG. 4 shows the relative intensity measured by total ion chromatography (TIC) and oligosaccharide composition of hFF aHS and iHS following enzymatic digestion as described in Example 2. A: TIC of hFF aHS; B: TIC of hFF iHS; C: composition of each fraction. Disaccharides are detected (peaks 1-4) and a substantial amount tetra- and hexasaccharides (in peaks 5-8).

FIG. 5 shows the structure determination of hFF aHS and iHS tetrasaccharides by MS/MS as described in Example 2. A: Tetrasaccharide in peak 5 (FIG. 4) at m/z 437; B: Tetrasaccharide in peak 6 (FIG. 4) at m/z 456; C: Tetrasaccharide in peak 7 (FIG. 4) at m/z 477; D: Tetrasaccharide in peak 8-1 (FIG. 4) at m/z 517; E: Hexasaccharide in peak 8-2 (FIG. 4) at m/z 685.

### DETAILED DESCRIPTION

The term “efficacy” of a treatment according to the invention can be measured based on changes in the course of disease in response to a use according to the invention. For example, the efficacy of a treatment according to the invention can be measured by a decrease in occurrence and severity of cardiovascular pathologies related to endothelial activation such as thrombosis, related stroke and the like. For another example, the efficacy of the treatment of proteolysis and inflammation mediated cell invasion in metastasis or cell proliferative disorder (e.g. cancer) encompasses inhibition or reduction of tumor foci, cell proliferative capacity and the like.

The term “pharmaceutically acceptable” refers to a carrier comprised of a material that is not biologically or otherwise undesirable.

The term “carrier” refers to any components present in a pharmaceutical formulation other than the active agent and thus includes diluents, binders, lubricants, disintegrants, fillers, coloring agents, wetting or emulsifying agents, pH buffering agents, preservatives and the like.

As used herein, “treatment” and “treating” and the like generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e., arresting its development; or relieving the disease, i.e., causing regression of the disease and/or its symptoms or conditions such as improvement or remediation of damage.

The term “subject” as used herein refers to mammals. For examples, mammals contemplated by the present invention include human, primates, domesticated animals such as cattle, sheep, pigs, horses, laboratory rodents and the like.

The term “thrombotic” disease or disorder comprises disorders or diseases involving the formation of a blood clot that blocks a blood vessel. Typically, thrombotic diseases or disorders includes heart attack, stroke, peripheral arterial occlusion, deep vein thrombosis, pulmonary embolism, atherosclerosis related diseases related to inflammation mediated endothelial activation such as lipid mediated endothelial activation.

The term “proteolysis and inflammation mediated cell invasion” diseases or disorders, include vascular endothelial inflammation as well as invasive cells such as cancer cells or trophoblasts. It also includes the angiogenesis and cell migration such as those occurring during ovulation and corpus lutum formation.

The term “infertility” includes ovulatory disorders, embryo implantation disorders such as extra-uterine implantation, endometrial receptivity disorders and placentation disorders. Broadly, infertility includes fertility decrease such as irregular ovulatory cycles (oligoovulation) or age induced fertility rate decrease. Infertility includes reproductive diseases linked to placentation resulting in pre-eclampsia, intrauterine growth retardation or arterial hypertension.

The term “ovulation” includes the follicular rupture and the oocyte delivery to the oviduct.

### Method of Preparation

The glycosaminoglycan compositions according to the invention may be prepared by a method a method according to the invention.

In one embodiment is provided a method for preparing a glycosaminoglycan composition comprising the following steps:
(a) Providing a follicular fluid sample;
(b) Purifying the said follicular fluid sample by ion exchange chromatography to eliminate the bulk of the proteins and recover the charged proteoglycans and glycoaminoglycans;
(c) Digesting the purifed proteoglycans and glycoaminoglycans obtained under step (b) with chondroitinase ABC to eliminate non-heparan sulfate species;
(d) Isolating of free glycoaminoglycans from high molecular weight proteoglycans by gel filtration;
(e) Recovering heparan sulfate glycoaminoglycan chains by β-eliminative cleavage on the isolated high molecular weight proteoglycans obtained under step (d);
(f) Removing proteins from the recovered heparan sulphate glycoaminoglycan chains fraction obtained under step (e) by phenol extraction;
(g) Recovering a glycosaminoglycan composition from the protein free fraction obtained under step (f) by ethanol precipitation.

Typically, the follicular fluid sample provided under step (a) is obtained from patients with ovulation induction for IVF treatment, with written informed consent, at the time of oocyte pickup.

Purifying step (b) is carried out to eliminate the bulk of the proteins to recover the charged proteoglycans and glycoaminoglycans. Typically, it can be carried out by ion exchange chromatography first on a DEAE-Sephacel and followed by a MonoQ ion exchange chromatography separation where the collected fractions are those binding to antithrombin (AT). Typically, the cut-off for the collection of those fractions is determined on the basis of detection by ¹²⁵I-AT ligand-binding assay such as described in De Agostini et al., 1994, J. Cell. Biochem. 54, 174-185.

The digesting step (c) is carried out to degradate the non-heparan sulphate glycosaminoglycans (chondroitin sulfate, dermatan sulfate and hyaluronan).

The isolating step (d) by gel filtration is carried out in order to remove free glycoaminoglycans from the high molecular weight proteoglycans. Typically step (d) is carried out on Sepharose CL4B.

In another aspect, the invention provides a method for preparing a glycosaminoglycan composition wherein the method further comprises the following steps after step (g):

(h) Isolating the anticoagulant (aHS) and non-anticoagulant (iHS) heparan sulphate fractions from the composition obtained under step (g) by antithrombin affinity gel chromatography.
(i) Recovering separately the two fractions obtained under step (h).

Typically, the isolation step (h) is carried out by (1) generating complexes of antithrombin with the aHS heparan sulphate fraction by addition of AT; (2) capturing the formed antithrombin complexes obtained under step (1) on a gel chromatography column that binds AT glycoconjugates such as a concanavalin A-Sepharose column; (3) dissociating the aHS-AT complexes obtained under step (2) for releasing aHS; and (4) recovering the iHS heparan sulphate fraction recovered in a flow-through of the column.

Examples illustrating the methods of the invention are further described in the Examples.

### Compositions

The invention provides pharmaceutical or therapeutic agents as compositions and methods for treating a patient, preferably a mammalian patient, and most preferably a human patient who is suffering from a medical disorder, and in particular a disorder selected from a thrombotic disease, a proliferative disease such as cancer, proteolysis and inflammation mediated tissue remodeling and cell invasion, and infertility.

In a particular embodiment, the invention provides a glycosaminoglycan composition according to the invention for use as a medicament.

The invention provides a glycosaminoglycan composition according to the invention for use in the treatment of a disorder selected from a thrombotic disease, a proliferative disease such as cancer, proteolysis and inflammation mediated tissue remodeling and cell invasion, and infertility.

Pharmaceutical compositions of the invention can contain at least one glycosaminoglycan composition according to the invention in any form described herein. Compositions of this invention may further comprise one or more pharmaceutically acceptable additional ingredient(s) such as alum, stabilizers, antimicrobial agents, buffers, coloring agents, flavoring agents, adjuvants, and the like.

The glycosaminoglycan composition according to the invention, together with a conventionally employed adjuvant, carrier, diluent or excipient may be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, or in the form of sterile injectable solutions for parenteral (including subcutaneous) use. Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed. Compositions according to the invention are preferably injectable.

Glycosaminoglycan compositions of this invention may also be liquid formulations including, but not limited to, aqueous or oily suspensions, solutions, emulsions, syrups, and elixirs. Liquid forms suitable for oral administration may include a suitable aqueous or non-aqueous vehicle with buffers, suspending and dispensing agents, colorants, flavors and the like. The compositions may also be formulated as a dry product for reconstitution with water or other suitable vehicle before use. Such liquid preparations may contain additives including, but not limited to, suspending agents, emulsifying agents, non-aqueous vehicles and preservatives. Suspending agent include, but are not limited to, sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid. Further materials as well as processing techniques and the like are set out in Part 5 of Remington's Pharmaceutical Sciences, 20^{th} Edition, 2000, Mack Publishing Company, Easton, Pa., which is incorporated herein by reference.

Solid compositions of this invention may be in the form of tablets or lozenges formulated in a conventional manner. For example, tablets and capsules for oral administration may contain conventional excipients including, but not limited to, binding agents, fillers, lubricants, disintegrants and wetting agents. Binding agents include, but are not limited to, syrup, accacia, gelatin, sorbitol, tragacanth, mucilage of starch and polyvinylpyrrolidone. Fillers include, but are not limited to, lactose, sugar, microcrystalline cellulose, maizestarch, calcium phosphate, and sorbitol. Lubricants include, but are not limited to, magnesium stearate, stearic acid, talc, polyethylene glycol, and silica. Disintegrants include, but are not limited to, potato starch and sodium starch glycollate. Wetting agents include, but are not limited to, sodium lauryl sulfate. Tablets may be coated according to methods well known in the art.

Injectable compositions are typically based upon injectable sterile saline or phosphate-buffered saline or other injectable carriers known in the art.

Compositions of this invention may also be formulated as suppositories, which may contain suppository bases including, but not limited to, cocoa butter or glycerides. Compositions of this invention may also be formulated for inhalation, which may be in a form including, but not limited to, a solution, suspension, or emulsion that may be administered as a dry powder or in the form of an aerosol using a propellant, such as dichlorodifluoromethane or trichlorofluoromethane. Compositions of this invention may also be formulated transdermal formulations comprising aqueous or non-aqueous vehicles including, but not limited to, creams, ointments, lotions, pastes, medicated plaster, patch, or membrane.

Compositions of this invention may also be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

Compositions of this invention may also be formulated as a depot preparation, which may be administered by implantation or by intramuscular injection. The compositions may be formulated with suitable polymeric or hydrophobic materials (as an emulsion in an acceptable oil, for example), ion exchange resins, or as sparingly soluble derivatives (as a sparingly soluble salt, for example).

Compositions of this invention may also be formulated as a liposome preparation. The liposome preparation can comprise liposomes which penetrate the cells of interest or the stratum corneum, and fuse with the cell membrane, resulting in delivery of the contents of the liposome into the cell. Other suitable formulations can employ niosomes. Niosomes are lipid vesicles similar to liposomes, with membranes consisting largely of non-ionic lipids, some forms of which are effective for transporting compounds across the stratum corneum.

The compounds of this invention can also be administered in sustained release forms or from sustained release drug delivery systems. A description of representative sustained release materials can also be found in the incorporated materials in Remington's Pharmaceutical Sciences.

### Mode of Administration

Compositions of this invention may be administered in any manner including intravenous injection, subcutaneous injection and oral route, but not limited to subcutaneous injection as currently used for heparin. Delivery methods for the composition of this invention include known delivery methods for heparin such as for example described in U.S. Pat. No. 4,654,327, U.S. Pat. No. 4,656,161, U.S. Pat. No. 5,853,749 and U.S. Pat. No. 5,633,226.

### Combination

According to the invention, glycosaminoglycan compositions and pharmaceutical formulations thereof can be administered alone or in combination with a co-agent useful in the oral treatment of thrombosis and of cell invasion such as metastatic invasion, e.g. for example a co-agent selected from compounds such as deoxycholic acid (Lee et al., 2008, Clin. Cancer Res., 14(9):2841-9; Kim et al., 2007, J. Control. Release., 123(2):155-63; Moazed et al., 2007, J. Pharmacol. Exp. Ther., 322(1):299-305).

The invention encompasses the administration of glycosaminoglycan compositions and pharmaceutical formulations thereof, wherein the glycosaminoglycan composition or the pharmaceutical formulation thereof is administered to an individual prior to, simultaneously or sequentially with other therapeutic regimens or co-agents useful in the treatment of proliferative disease such as metastatic cell invasion (e.g. multiple drug regimens), in a therapeutically effective amount. Glycosaminoglycan compositions or the pharmaceutical formulations thereof that are administered simultaneously with said co-agents can be administered in the same or different composition(s) and by the same or different route(s) of administration.

### Patients

In an embodiment, patients according to the invention are patients suffering from disorders related to e.g. disorders such as a thrombotic disease, a proliferative disease such as cancer, proteolysis and inflammation mediated tissue remodeling and cell invasion, and infertility. In particular, the patients according to the invention are suffering from thrombosis.

In another particular embodiment, the patients according to the invention are men or post-menopausal women, notably at risk of thrombosis (e.g. suffering from arteriosclerosis). In another particular embodiment, the patients according to the invention are female suffering from infertility, notably implantation disorders.

### Use According to the Invention

In an embodiment, the invention provides a method for preparing a glycosaminoglycan composition comprising according to the invention.

In another embodiment of the invention is provided a use of a glycosaminoglycan composition according to the invention for the preparation of a pharmaceutical composition for the prevention or treatment of a thrombotic disease, a cell proliferation disorder such as a proliferative disease e.g. cancer, proteolysis and inflammation mediated tissue remodeling and cell invasion, and infertility.

In a further embodiment, the invention provides a use of a glycosaminoglycan composition according to the invention for the preparation of a pharmaceutical composition for the prevention or treatment of thrombosis in a male or a post-menopausal female.

In another embodiment, the invention provides a method of prevention and/or treatment of a disease comprising the administration of a therapeutically effective amount of a glycosaminoglycan composition according to the invention in a mammal in need thereof and wherein the disease is selected from a thrombotic disease, a cell proliferation disorder such as a proliferative disease e.g. cancer, proteolysis and inflammation mediated tissue remodeling and cell invasion, and infertility.

In another embodiment, the invention provides a method of monitoring the follicular rupture at ovulation comprising the step of:
(a) Providing a blood sample from a female patient;
(b) Measuring the amount of anticoagulant heparan sulfate (aHS) in the sample provided under step (a);
(c) Comparing the amount of aHS measured under step (b) with an amount of aHS standard for a female in a non-ovulatory period.

In a further embodiment, the measuring step (b) can be performed through an anticoagulant test as described in Example 3.

In another further embodiment, the method of monitoring ovulation according to the invention may be conducted over a period of days in an ovulation cycle on blood samples from a female patient to detect a change in the concentration of aHS indicative of the actual event of ovulation (follicular rupture). In this case, the method comprises several cycles of steps (a) to (c), wherein the amount of aHS standard for a female in a non-ovulatory period under step (c) is the amount of aHS for the same patient measured in a non ovulatory period, preferably the preceding day.

In another further embodiment, the invention provides a method of monitoring ovulation, in particular follicular rupture, according to the invention wherein the female patient is a female suffering from infertility such as ovulatory disorder, implantation disorders, scarce fertility periods or female following a fertility check.

A method of monitoring ovulation and kits according to the invention are useful in determining the time of maximum fertility or the fertile period in a mammalian ovulation cycle, wherein testing is conducted over a period of days in an ovulation cycle on blood samples.

Alternatively, it can be of great importance to determine the monitor ovulation in order to ensure that fertilization occurs and that offspring are produced. This determination is useful to owners of pets, such as cats and dogs, as well as to breeders of livestock and particularly to breeders of race horses or cattle. While the monitoring of ovulation is of importance in breeding animals; of even greater importance is the ability to monitor whether and when a human female ovulates so that her chances of producing desired offspring may be increased and/or ovulatory disorders detected.

The method and kits of the invention have the advantage that it allows, with a high degree of accuracy, the determination of an ovulation day (through the detection of follicular rupture), and hence a fertile period, within a cycle. When needed for contraception purposes, this leads to a method of prediction of the fertile period which requires a minimal period of abstinence from unprotected intercourse within any given menstrual cycle.

A test kit for detecting ovulation, the kit comprising:

(a) At least one blood sample testing device that provides a readable signal proportional to the aHS concentration in a blood sample;
(b) An electronic monitor having reading means to read the readable signal obtained under step (a) and incorporating computer means to interpret the readable signals and to determine therefrom in conjunction with data from previous blood sample tests whether the event of ovulation in the current cycle has just occurred.

A method of monitoring ovulation according to the invention may be coupled to other methods of monitoring the onset of the fertile period or ovulation in female mammals such as for example monitoring at least one analyte selected from the group consisting of human chorionic gonadotrophin (hCG), luteinizing hormone (LH), β-estradiol, prolactin, follicle stimulating hormone (FSH) and metabolites thereof in a body fluid.

According to another embodiment of the invention is provided an isolated glycosaminoglycan composition comprising: (a) at least about 2% 3-O-sulfated glucosamines (GlcNS3S or GlcNS3S6S); and (b) about 0.5 to 2 sulfate per disaccharide unit.

According to a further embodiment, is provided an isolated glycosaminoglycan composition according to the invention, further comprising at least about 20% of non-sulfated disaccharides and at least about 60% sulfated disaccharides.

According to another further embodiment, is provided an isolated glycosaminoglycan composition according to the invention, further comprising at least about 10% 2-O-sulfated uronic acids (IdoA2S) and at least 70% non-sulfated uronic acids.

According to another further embodiment, is provided an isolated glycosaminoglycan composition according to the invention, further comprising at least about 15% 6-O-sulfated glucosamines (GlcNAc6S or GlcNS6S or GlcNS3S6S).

According to another further embodiment, is provided an isolated glycosaminoglycan composition according to the invention, wherein the composition contains at least about 10% glycosaminoglycans, with an anti-Factor Xa specific activity of at least 50 UI/mg.

According to another further embodiment, is provided an isolated glycosaminoglycan composition according to the invention, wherein the composition contains glycosaminoglycans having a chain size of at least about 10 kDa and wherein those chains contain at least two 3-O-sulfated glucosamines.

According to another further aspect of the invention, is provided an isolated glycosaminoglycan composition according to the invention, wherein the composition contains at most about 30% 3-O-sulfated glucosamines.

According to another further aspect of the invention, is provided an isolated glycosaminoglycan composition according to the invention, wherein the composition contains at most about 2 sulfates per disaccharide.

According to another further aspect of the invention, is provided an isolated glycosaminoglycan composition according to the invention, wherein the composition contains at most about 80% sulfated disaccharides and at most about 40% non-sulfated disaccharides.

According to another further aspect of the invention, is provided an isolated glycosaminoglycan composition according to the invention, wherein the composition contains at most about 30% 2-O-sulfated uronic acids and at most about 90% non-sulfated uronic acids.

According to another further aspect of the invention, is provided an isolated glycosaminoglycan composition according to the invention, wherein the composition contains at most about 50% 6-O-sulfated glucosamines.

According to another further embodiment, is provided an isolated glycosaminoglycan composition according to the invention, wherein the composition contains glycosaminoglycans having a chain size of at most about 50 kDa and wherein those chains contain at most fifteen 3-O-sulfated glucosamines.

According to another further embodiment of the invention is provided an isolated glycosaminoglycan composition according to the invention comprising:

(a) 2 to 30% of 3-O-sulfated glucosamines;
(b) 0.5 to 2 sulfate per disaccharide unit;
(c) 20 to 40% of non-sulfated disaccharides and 60 to 80% of sulfated disaccharides;
(d) 10 to 30% of 2-O-sulfated uronic acids and 70 to 90% non-sulfated uronic acids;
(e) 15 to 50% of 6-O-sulfated glucosamines;
(f) at least about 10% of glycosaminoglycans with an anti-Factor Xa specific activity of at least 50 UI/mg; wherein the composition contains glycosaminoglycans having a chain size of 10 to 50 kDa and wherein those chains contain two to fifteen 3-O-sulfated glucosamines.

A glycosaminoglycan composition according to the invention can be used for the treatment of pathological conditions involving proteolysis and inflammation mediated cell invasion, including: (i) the treatment and prevention of thrombotic disorders, (ii) the treatment and prevention of acute or chronic vascular inflammation, (iii) the treatment of invasive cancer, through the prevention of dissemination and (iv) the treatment of tumors via inhibition of tumor induced angiogenesis.

In a particular embodiment, is provided a glycosaminoglycan composition according to the invention useful in the prevention and/or treatment of thrombotic disorders comprising:

(a) 2 to 30% of 3-O-sulfated glucosamines;
(b) 0.5 to 2 sulfate per disaccharide unit;
(c) 10 to 30% of 2-O-sulfated uronic acids and 70 to 90% non-sulfated uronic acids;
(d) 15 to 50% of 6-O-sulfated glucosamines;
(e) at least about 10% of glycosaminoglycans with an anti-Factor Xa specific activity of at least 50 UI/mg; wherein the composition contains two to fifteen 3-O-sulfated glucosamines per heparan sulfate chain.

In another particular embodiment, a glycosaminoglycan composition according to the invention useful in the prevention and/or treatment of inflammatory disorders comprising:

(a) 2 to 30% of 3-O-sulfated glucosamines;
(b) 0.5 to 2 sulfate per disaccharide unit;
(c) 20 to 40% of non-sulfated disaccharides and 60 to 80% of sulfated disaccharides;
(d) 10 to 30% of 2-O-sulfated uronic acids and 70 to 90% non-sulfated uronic acids;
(e) 15 to 50% of 6-O-sulfated glucosamines; wherein the composition contains glycosaminoglycans having a chain size of 10 to 50 kDa and wherein those chains contain two to fifteen 3-O-sulfated glucosamines.

Glycosaminoglycan compositions according to the invention may be useful in the prevention of thrombotic risk as seen in cycling women. The present composition according to the invention is useful to provide arterial protection as found in cycling women. Further, the use of compositions according to the invention compounds is very attractive because they are devoid of adverse effects of known anticoagulants.

Glycosaminoglycan compositions according to the invention may be further useful in the treatment of ovulatory disorders, for example by preventing clotting of follicular fluid, favouring its fluidity at ovulation and/or ensuring the correct delivery of the oocyte to the oviduct where it fertilises.

Glycosaminoglycan compositions according to the invention may be further useful in favouring correct implantation, for example by modulating the adhesion of the embryo by virtue of its ability to prevent adhesion to epithelial cells in the oviduct, thereby preventing extra-uterine implantation.

Examples illustrating the invention will be described hereinafter in a more detailed manner and by reference to the embodiments represented in the Figures.

### EXAMPLES

The following abbreviations refer respectively to the definitions below:

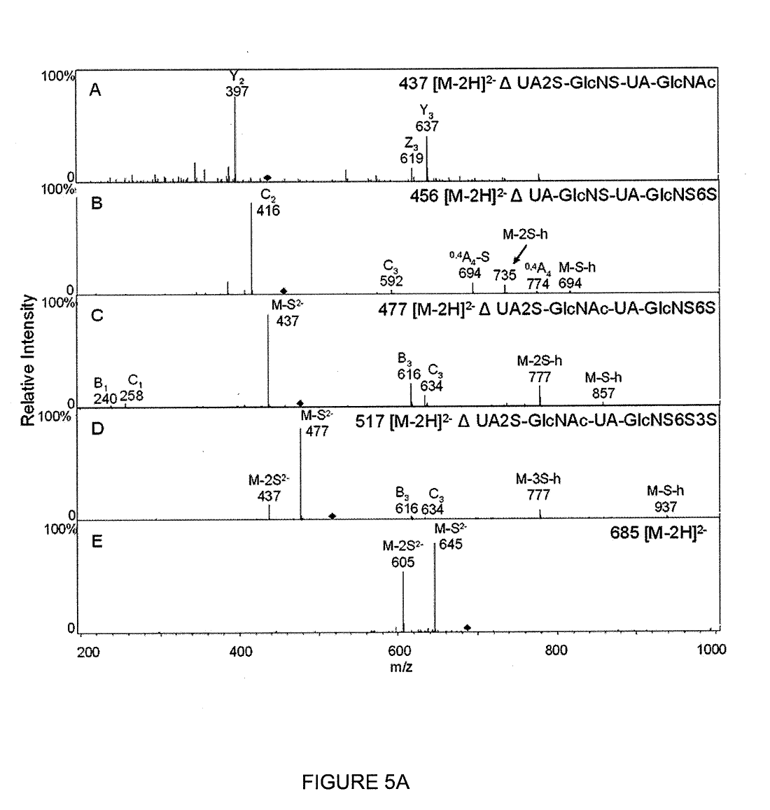

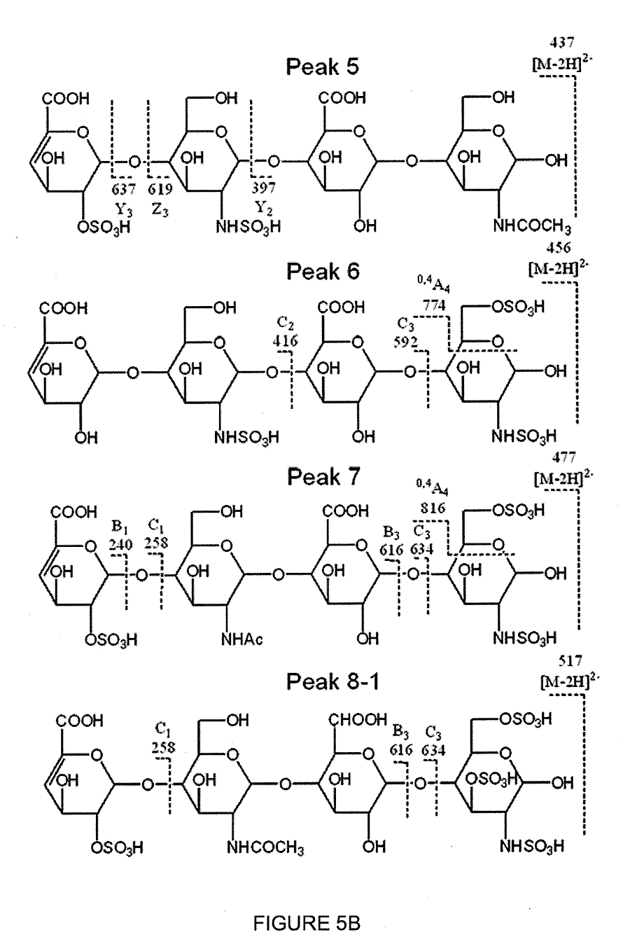

cm (centimeter), cpm (count per minute), Da (Dalton), h (hour), IU (Iternational Unit), K_{D} (dissociation constant of a complex), kHz (Kilohertz), Kₒₙ, (rate of association M⁻¹sec⁻¹), K_{off} (dissociation rate sec⁻¹), μg (microgram), mg (milligram), mHz (megahertz), min (minute), ml (milliliter), mm (millimeter), mM (millimolar), nM (nanomolar), nm (nanometer), psi (Pounds per Square Inch), sec (second), V (volt), AT (Antithrombin), CHAPS (3-[(3-cholamidopropyl)-dimethylammonio]-1-propanesulfonate), DEAE (Diethylaminoethyl), FPLC (fast performance liquid chromatography), GAG (Glycoaminoglycan), GC (Granulosa Cell), hFF (human follicular fluid), HSPG (heparan sulphate proteoglycan), IVF (in vitro fertilization), LC (liquid chromatography), MOPS (3-[N-morpholino]propanesulfonic acid), MS (mass spectrometry), MW (Molecular Weight), NMR (Nuclear magnetic Resonance), OD (Optical Density), PBS (Phosphate buffered saline), PSGL-1 (P-selectin glycoprotein ligand), PT (prothrombin time), TIC (Total Ion Chromatography), TT (thrombin time). Percentages are all expressed as signal intensity of a peak as compared to the total signal (FIG. 4, FIG. 5), and as w/w for the percentage aHS and iHS. For coagulation parameters, percentages are related to activity units/activity units of a reference sample or as clotting time as compared to the clotting time of a reference sample.

### General Procedures & Conditions

The method according to the invention is a method of purification of a glycosaminoglycan composition from a follicular fluid sample may be for example achieved through the following conditions:

Purifying step (b) by ion exchange chromatography is first performed on a DEAE-Sephacel Column in 50 mM phosphate buffer containing 0.15 M NaCl and step eluted with 2M NaCl, so that most of the proteins are eliminated from hFF.

Purifying step (b) by ion exchange chromatography is then secondly performed by a MonoQ ion exchange chromatography where fractions eluting between 0.96 M and 1.38M NaCl are collected. The cutoff for the collection of those fractions is determined on the basis of detection by ¹²⁵I-AT ligand-binding assay such as described in De Agostini et al., 1994, J. Cell. Biochem. 54, 174-185.

The isolating step (d) by gel filtration is carried out on a Sepharose CL4B gel and the fractions eluting at Kₐᵥ (relative elution position in gel filtration) 0.0-0.1 are collected. Free heparin (liquemin) is excluded from the collection (Kₐᵥ=0.6).

In order to monitor the purification steps, the following detection tools may be used:

Detection of proteins: OD₂₈₀ₙₘ
Detection of glycosaminoglycans: Alcian Blue
Detection of aHSPG: ¹²⁵I-AT-ligand binding assay

The obtained glycosaminoglycan composition obtained by the above method may be further fractioned through the further steps (h) and (i) described above, according to their according to their binding affinity to AT. Typically, the purification of 60 ml of hFF yields about 200 μg glycosaminoglycan composition according to the invention containing 50% aHS and 50% iHS.

The antithrombotic activity of the glycosaminoglycan compositions according to the invention may be assayed in an in vivo model of oophorectomized mice with or without treatment by aHS or iHS followed by a thrombotic challenge such as described in Zadelaar et al. Arterioscle. Thromb. Vasc. Biol. 2007; 27:1706-21.

### Example 1

### Preparation of a Composition According to the Invention

Human follicular fluid samples were collected as described below. The coagulation parameters of the citrated hFF collected from IVF patients in which ovulation was induced with gonadotrophins was measured according to the protocol below.

The isolation of a glycosaminoglycan composition according to the invention from native hFF required extensive purification as native hFF contains an average of 40 mg protein/ml, similar to plasma. It was thus necessary to include two consecutive ion exchange chromatographies (DEAE-Sephacel column and MonoQ column) as described below to eliminate the bulk of the proteins.

The highly charged proteoglycans and GAGs recovered were digested with chondroitinase ABC to eliminate non-HS species.

High molecular weight HSPG were isolated by gel filtration (Sepharose CL4B column) as described below. A final purification step allowed to isolate heparan sulphate chains from HSPG after β-eliminative cleavage from the PG core protein, the HS fraction was purified by phenol extraction and concentrated by ethanol precipitation. This allowed to produce pure HS chains released from the purified HSPG and devoid of free endogenous GAGs or of heparin contamination.

### Collection of hFF and Human Granulosa Cells

Human hFF was collected from female patients. The patients were scheduled for IVF treatment of infertility and ovulation was induced with gonadotrophins. Follicular fluid was recovered at the time of oocyte pickup, after removal of the oocytes for IVF. The hFF samples were pooled for each patient, carefully avoiding samples containing washing solution (G-MOPS containing 2.5 IU/ml liquemin), and cleared by centrifugation, at low speed to remove cells (800×g, 10 min, 20° C.) and subsequently at high speed to remove insoluble aggregates (13,000×g, 30 min, 4° C.). The first, low speed centrifugation allowed the recovery of human granulosa cells. The supernatant afforded by centrifugation was called native hFF and used in the experiments described.

For clotting assays, hFF samples were quickly taken from the hFF pool before centrifugation and supplemented with 1/10 volume of sodium citrate (0.13 M), to prevent spontaneous activation of the coagulation cascade. Citrated samples were purified by centrifugation as the rest of the hFF pool. The extent of contamination by blood was evaluated by counting the red blood cells present in the hFF samples and only samples with blood contamination below 1% were used for coagulation tests. Samples were stored frozen at −80° C. in aliquots until used.

### Ion Exchange Batch Chromatography on DEAE Sephacel

hFF obtained after collection was admixed with 0.6% 3-[(3-cholamidopropyl)-dimethylammonio]-1-propanesulfonate (CHAPS) (w/v) and 0.5 ml DEAE Sephacel gel equilibrated in PBS (sodium phosphate 50 mM at pH 7.4 with 150 mM NaCl) was added per 1 ml hFF. The slurry was mixed on a rotary mixer for 3 h at 4° C. and subsequently washed on glass filter with PBS until no more proteins eluted, as detected by Coomassie blue staining in the effluent. The gel was washed successively with two gel volumes of PBS containing 0.6% CHAPS, with 10 volumes of PBS, with two column volumes of sodium acetate 50 mM pH 5.0 and with PBS to restore the pH at 7.4. The gel was then packed in a column and eluted with a two step NaCl gradient in PBS consisting of a first linear gradient of 0.15-1.0 M NaCl, followed by a wash with 1.0 M NaCl and a second linear gradient of 1.0-2.0 M NaCl followed by a wash with 2.0 M NaCl. The aHSPG was followed by ¹²⁵I-AT ligand-binding assay and proteins by absorbance at OD₂₈₀ₙₘ and glycosaminogylcans (GAGs) by Alcian Blue (Fluka Chemical Corp., Milwakee, Wis., U.S.A.) assay using heparin as standard (Bjornsson, 1998, Anal. Biochem. 256:229-237). The aHSPG was eluted as a major peak at 0.71-0.88 M NaCl. Fractions containing proteins and GAGs were pooled, dialyzed against PBS and concentrated on an Amicon concentrator (Amicon plastics, Houston, Tex., U.S.A.) using a PM 30 membrane. Insoluble material was removed from the concentrate by filtration on a 0.22 μm pore size Millex membrane (Millipore Corp., Bedford, Mass., U.S.A.).

### Ion Exchange Chromatography on Mono Q Resin

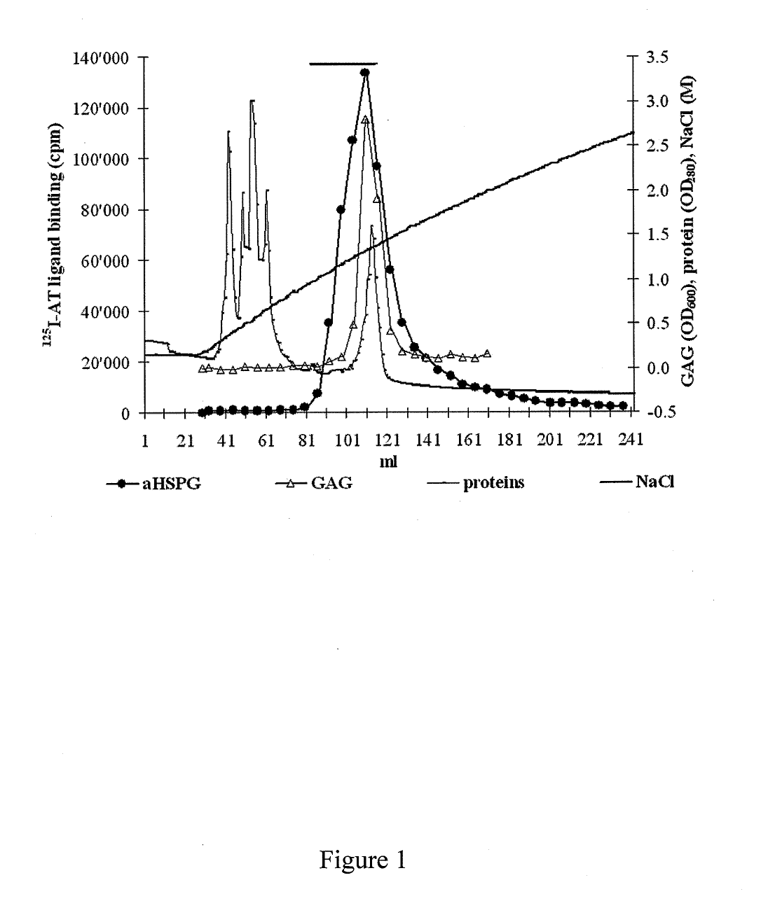

The sample purified by the above ion exchange chromatography was then purified on a MonoQ ion exchange column (diameter 10 mm, 10 ml gel) and eluted using a FPLC system (Pharmacia, Uppsala, Sweden). The loading buffer was PBS and, after washing with PBS and with two column volumes of 50 ml sodium acetate (pH 5.0), the bound material was eluted in PBS with a linear gradient (130 ml of 0.15 M to 3.0 M NaCl), at a flow rate of 2 ml/min. Elution was followed by absorbance at 280 nm for proteins, by Alcian Blue assay for GAGs and by ¹²⁵I-AT ligand-binding assay for aHSPGs. Pooled proteoglycan fractions were dialyzed and concentrated on an Amicon concentrator as previously described. All of the bound aHSPG and of the GAGs eluted together with a minor amount of protein, as a single proteoglycan peak comprised between 0.96 and 1.38 M NaCl (FIG. 1). These fractions were pooled for further purification (bar).

Non-HS GAGs (chondroitin sulfate, dermatan sulfate and hyaluronan) were degraded with chondroitinase ABC.

### Gel Filtration Chromatography on Sepharose CL4B

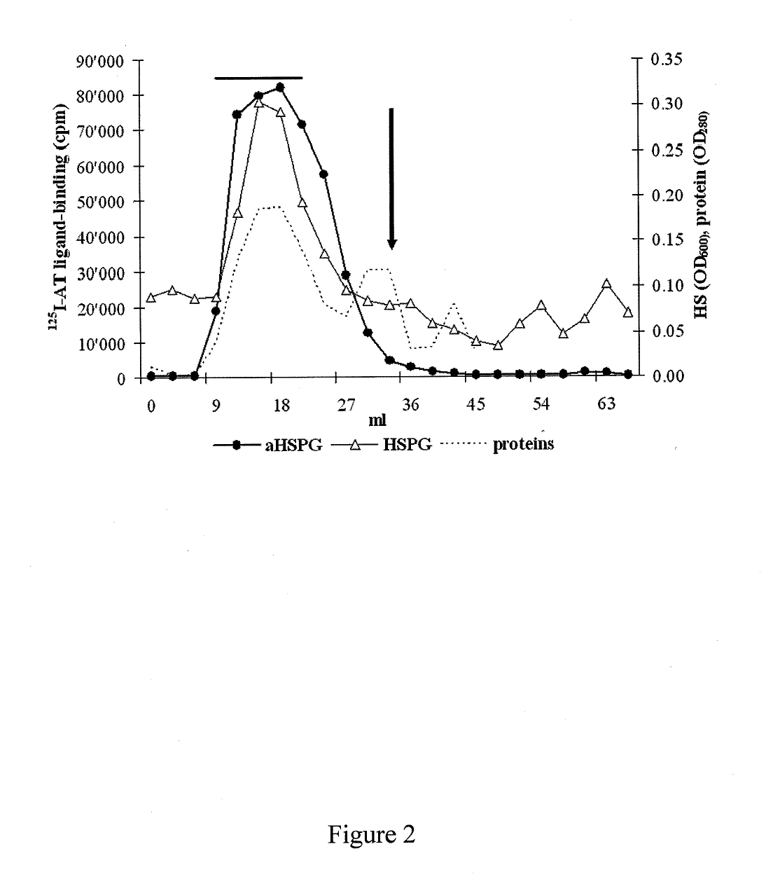

The high MW HSPGs were fractionated by gel filtration on Sepharose CL4B column (0.9×63 cm) in PBS. The inclusion volume of the column was determined using Dextran Blue (Pharmacia, Kₐᵥ=0) followed by OD₂₈₀ₙₘ and 2 M NaCl (Kₐᵥ=1) followed by conductivity, and the elution position of GAG chains was determined using heparin (Diosynth) cleaved from residual peptides by β-elimination such as described in Shworak et al., 1994, J. Biol. Chem. 269, 24941-24952 (Kₐᵥ=0.75) and with heparin (Liquemin, Kₐᵥ=0.62). The elution of hFF HS was followed by Alcian Blue, that of aHSPGs by ¹²⁵I-AT ligand-binding assay and proteins were followed by absorbance at 280 nm (OD₂₈₀ₙₘ) such as shown on FIG. 2. The major high MW HSPG peak containing protein, GAG and aHSPG, that eluted as at Kₐᵥ 0.0 to 0.1 and was clearly separated from free GAGs. Fractions eluting at Kₐᵥ 0-0.1 were pooled for further analysis (indicated by a bar). Minor HS peaks of lower MW representing free GAG were discarded. For comparison, heparin (MWₐᵥ 16,400) eluted at Kₐᵥ 0.6 (arrow) clearly separated from the HSPG peak (FIG. 2).

Detection of aHSPG in hFF by 125I-AT-Ligand Binding Assay

Briefly, native hFF was filtered on a 0.22 μm Millex filter to remove insoluble material and loaded on a nitrocellulose membrane using a dot-blot apparatus. The amount of protein loaded per well was kept below 20 μg, to avoid saturation of the membrane, which was subsequently saturated in blotto buffer (5% non-fat dry milk in 10 mM Tris-HCl pH 7.4, 150 mM NaCl) for 30 min at room temperature and incubated for 2 h in the same buffer containing ¹²⁵I-AT (1×10⁶ cpm/ml, ˜1 nM). After 5 washes in the same buffer, the membrane was exposed for autoradiography and radioactivity quantified in a γ-counter. Measurements were done in triplicate and the results expressed as cpm/ml sample loaded onto the membrane. For controls, we used conditioned media from the aHSPG-positive reference cell line LTA (aHSPG positive reference fibroblastic cell line as described in De Agostini et al., 1994, J. Cell. Biochem. 54:174-185). In this assay, the aHSPG are bound to the nitrocellulose membrane through their core protein, and any cleavage occurring between the linkage region of the HS chain and the AT-binding site results in the loss of AT-binding due to the releases of HS. Heparin lyase III cleaves undersulfated regions of HS at glycosidic linkages containing a nonsulfated uronic acid.

### Example 2

### Further Characterization of the Properties of Purified hFF Fractions

Pure hFF HS obtained after release of the GAGs from HSPG recovered after gel filtration were finally fractionated according to their AT-affinity into aHS and iHS fractions as described above.

### a) AT-Affinity

After incubation of HS with AT to allow the formation of aHSAT complexes, the latter were isolated from iHS by binding of the AT moiety to concanavalin A-Sepharose. The aHS was eluted with 1M NaCl. It revealed that 50.4% of the HS chains bind to AT. Therefore, the hFF HS was found to contain 50.4% of aHS, a much higher amount than in heparin that typically contains about 30% AT-binding chains (Table 1 below).

TABLE 1

| Sample | aHS | iHS |
|---|---|---|
| μg HS/60 ml hFF | 107.1 ± 16.01 | 109.1 ± 30.1 |
| μg HS/ml hFF | 1.8 ± 0.2 | 1.8 ± 0.5 |
| % HS | 50.4 ± 5.1 | 49.6 ± 5.1 |

mean ± sem, n = 6

The purification yield provided enough material for physico-chemical characterization of hFF aHS and iHS.

### b) Molecular Size Distribution

The molecular size distribution of hFF aHS and iHS was determined by Azure A stained PAGE as describe below and showed similar size distribution of aHS and iHS with a modal MW of about 30,000 Da.

### c) Content in Highly Sulphated Domains

hFF, aHS and iHS fractions were with heparin lyase I and III and analyzed by PAGE stained with silver enhanced Azure as described below in order to determine the content in highly sulphated domains. The gel banding obtained with heparin lyases digestion shows that heparin lyases I and III leave digestion resistant fragments, suggesting that they contain 3-O-sulfated disaccharides that block heparin lyases cleavage. Thus, aHS and iHS seem to have comparable high sulfate and low sulfate domain structures.

d) 1D-¹H NMR structure

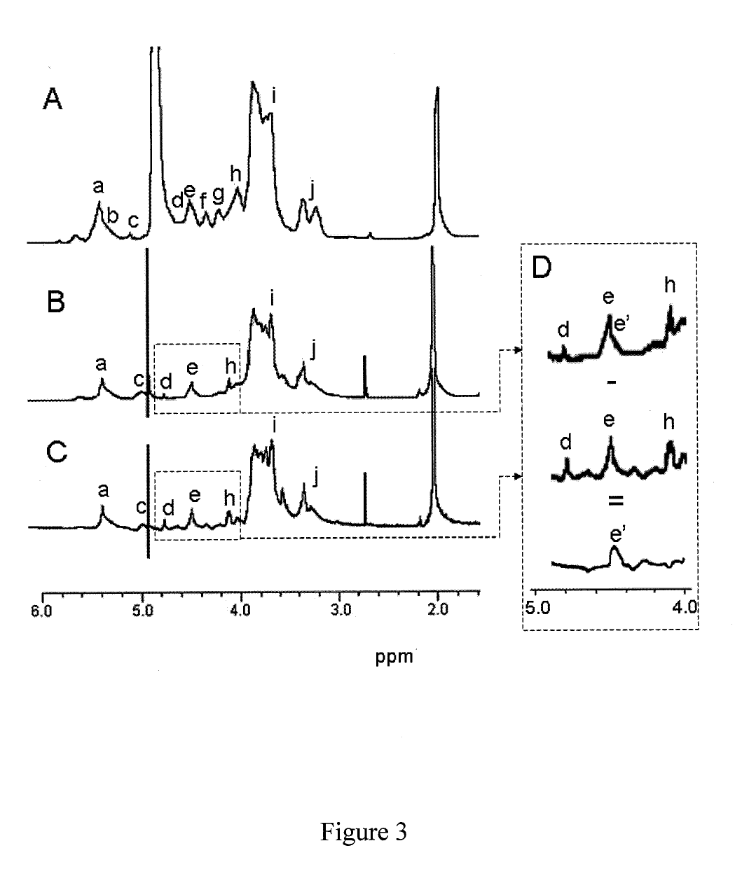

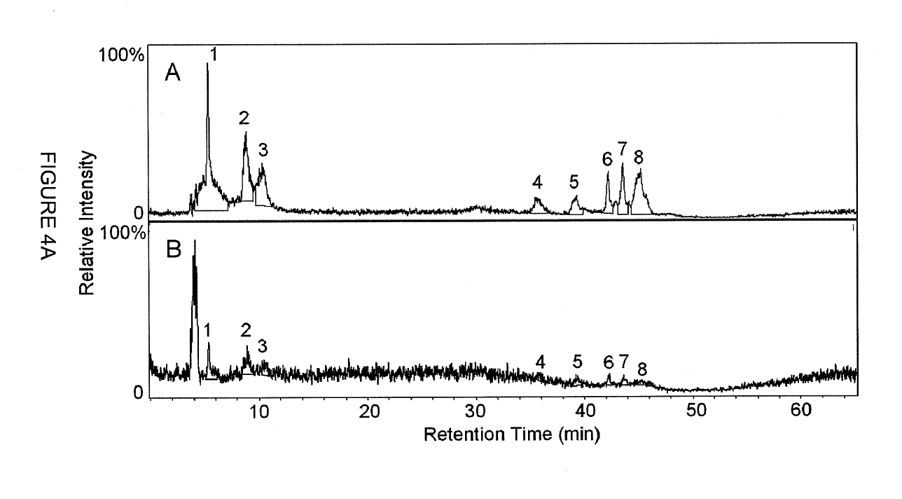

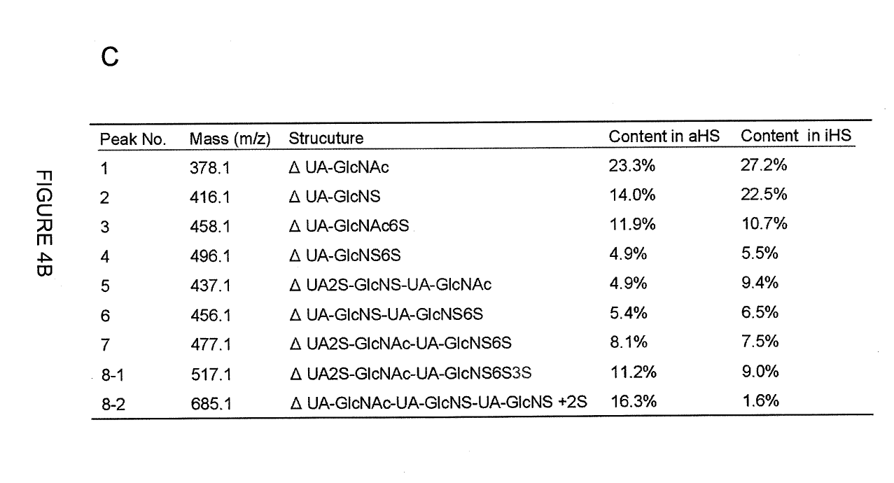

The structures of standard commercial HS and of the aHS and iHS fractions were analyzed by 1D-¹H-NMR according to the procedure below. The obtained spectra are presented on FIG. 3 and show that, compared to HS standard (FIG. 3A), hFF aHS (FIG. 3B) and iHS (FIG. 3C) present a typical pattern of HS with a lower content of residues containing 2-O-sulfated iduronic acid (IdoA2S), iduronic acid (IdoA) and 6-O-sulfated glucosamine (peaks b, c, d, f, g). hFF aHS and iHS also have higher peaks of H4,5,6 of GlcNAc (i) than standard HS, with the highest peak in iHS (FIG. 3). In consequence, the relative content in non-sulfated GlcNAc residue is iHS>aHS>standard HS.

In addition, in both aHS and iHS, the signal for 3-O-sulfated glucosamine residues (H-3 GlcNS3S or H-3 GlcNS3S6S) was observed near the signal of H-1 of GlcA, with minimal overlapping, demonstrating that aHS and iHS both contain 3-O-sulfated glucosamines.

It is noteworthy that the standard HS has higher degree of sulfation than both aHS and iHS and shows a signal for 3-O-sulfated glucosamines but is devoid of anticoagulant and anti-Xa activity. This is the first report of ¹H NMR structural analysis of human HS showing the presence of 3-O-sulfated glucosamine. Human liver HS ¹H NMR has been analyzed showing a spectrum comparable to that of porcine intestinal mucosa but without detectable signal for 3-O-sulfated glucosamine (Antonaccio et al, 1993, J. Pharmacol. Exp. Ther. 266:125-132).

### e) Composition in Oligosaccharides

The composition in oligosaccharides of hFF aHS and iHS fractions after enzymatic digestion by heparin lyase has been followed by total ion chromatography (TIC) as described below. The TIC of aHS and iHS digestion products are shown on FIGS. 4A and 4B, respectively, indicating the presence of not only disaccharides (peaks 1-4) but also of a substantial amount tetra- and hexasaccharides in peak 5, 6, 7.

The chemical structure of hFF aHS and iHS fractions was then further characterized by capillary HPLC/MS followed by MS/MS of the samples extensively digested by heparin lyase I, II and III as described below (FIG. 5). It was found that the four tetrasaccharides (FIG. 4C FIG. 5) contained 2-3 or 4 sulfate groups. Both aHS and iHS contained substantial amounts (11.7% and 9.0%, respectively) of the tetrasulfated tetrasaccharide (8-1) with a GlcNS6S3S in its reducing end that explains its resistance to heparin lyase digestion (FIG. 4C FIG. 5). This 3-O-sulfated tetrasaccharide is more abundant in aHS where it is the predominant tetrasaccharide. In addition, aHS contains high amounts of a tetrasulfated hexasaccharide (FIG. 5E) that could be due to the presence of at least one 3-O-sulfated glucosamine residue.

The relative abundance of 3-O-sulfated glucosamines detected in hFF iHS is in contrast to previously published evidence showing that 3-O-sulfated glucosamine residues were usually about 10-fold less abundant in iHS as in aHS (Kojima et al., 1992, J. Biol. Chem. 267:4859-4869; Marcum, et al., 1986, J. Biol. Chem. 261:7507-7517; De Agostini et al., 1990, Proc. Natl. Acad. Sci. U.S.A. 87:9784-9788). Despite their lack of affinity for AT, 1HS bear 3-O-sulfated glucosamines, presumably in sequences different from the AT-binding pentasaccharide found in aHS suggesting that these 3-O-sulfated iHS might have different biological activities.

### Determination of Average Molecular Weight (Mₐᵥ) of hFF aHS and his

Pure aHS and iHS obtained above were run on non-denaturing PAGE to estimate their MW as previously described (Hosseini et al., 1996, J. Biol. Chem. 271:22090-22099): aHS and iHS were subjected to electrophoresis on polyacrylamide gradient (11%-22%) gels without SDS, in buffer containing 0.1 M NaCl and stained using Azure A. GAG molecular weight standards were heparin (MWₐᵥ 16,400), chondroitin sulfate A (MWₐᵥ 21,600) and HS I (MWₐᵥ 15,500). HS or standard GAG sample (5 μg) was loaded in each lane and migration profiles were scanned and analyzed using the Aida software (Raytest, Isotopenmessgeräte GmbH, Straubenhardt, Germany). hFF HS molecular weight was determined by extrapolation from the regression of the standard GAG modal R_{F} and log MW (log MW=−27821 R_{F}+33969).

### Glycosidases Digestions

The glycosidases were heparinase lyase I (E.C. 4.2.2.7), II and III (E.C.4.2.2.8), and chondroitinase ABC (Sigma, ST Louis, Mo., U.S.A.). Heparin lyase I (heparinase) preferentially cleaves heparin, heparin lyase II cleaves heparin and HS equally, and heparin lyase III (heparitinase) preferentially cleaves HS. The heparin lyases used were either purified from Flavobacterium heparinum (Seikagaku, Tokyo, Japan) or were recombinant heparin lyases, a generous gift from Jian Liu (Chapel Hill University, NC, U.S.A.) (Chen et al., 2005, J. Biol. Chem. 280:42817-42825). Digestion with heparin lyases was done in phosphate buffered saline solution pH 7.4, using 20 mU/ml GAG and incubations at 37° C. for 2 h followed by a second addition of enzyme and overnight incubation at 37° C. Digestion with chondroitinase ABC was done in similar conditions, with an enzyme concentration of 0.1 U enzyme/ml substrate. Digestion with recombinant heparin lyases was done in 50 mM sodium acetate buffer (pH 7.0), using 50 μg/ml enzyme with 200 μg/ml substrate.

### 1D-1H-NMR Analysis

¹H-NMR was performed on Bruker 800 spectrometer with Topsin 2.0 software. Commercial HS (from porcine intestine, Celsus Co.), aHS and iHS 200 μg were each dissolved in 0.5 ml ²H₂O (99.996%, Sigma, Co. St. Louis, Mo.) and freeze-dried repeatedly to remove the exchangeable protons. The samples were re-dissolved in 0.3 ml ²H₂O and transferred to an NMR Shigemi tube (Sigma). The operation conditions for spectra were as follows: frequency, 800 MHz; wobble sweep width, 20 MHz; filter width, 125 KHz; pre-scan delay, 6 μs; transmitter frequency offset, 4.704 ppm; temperature, 300 K. The water resonance was suppressed by selective irradiation during the relaxation delay.

### LC-MS and MS/MS Analysis of hFF aHS and iHS Digested by Heparin Lyases

The aHS and iHS samples (30 μg, respectively) were incubated in 10 μl 50 mM sodium phosphate buffer pH 7.0 with heparin lyase I, II and III (10 m-units, Sigma Chemicals, St Louis, Mo., U.S.A.) at 37° C. for 10 h. The products were heated in a boiling water bath for 10 min. to halt the reaction. The denatured protein was removed by centrifugation at 12,000×g for 10 min. LC MS analyses were performed on Agilent 1100 LC/MSD instrument (Agilent Technologies, Inc. Wilmington, Del., U.S.A.) equipped with an ion trap, binary pump and a UV detector. The column was a 5 μm Agilent Zorbax SB-C18 (0.5×250 mm) from Agilent Technologies. Eluent A was water/acetonitrile (85:15), v/v and eluent B was water/acetonitrile (35:65) v/v. Both eluents contained 12 mM tributylamine and 38 mM NH₄OAc and their pH was adjusted to 6.5 with HOAc. The product mixtures of aHS and iHS (5 μl, respectively) were injected by auto-sampler. A gradient of 0% B for 15 min, and 0-50% B over 45 min. was used at a flow rate of 10 μl/min. Mass spectra were obtained using an Agilent 1100 series Classic G2445D LC/MSD trap (Agilent Technologies, Inc. Wilmington, Del., U.S.A.). The electrospray interface was set in negative ionization mode with the skimmer potential −40.0 V, capillary exit −20.0 V and a source temperature of 325° C. to obtain maximum abundance of the ions in a full scan spectrum (150-1500 Da, 10 full scans/s). Nitrogen was used as a drying (5 liters/min) and nebulizing gas (20 psi). Auto MS/MS was turned on in these experiments using an estimated cycle time of 0.07 min. Total ion chromatograms (TIC) and mass spectra were processed using Data Analysis 2.0 (Bruker software).

### Example 3

### Characterization of the Anticoagulant Activity of a Composition According to the Invention

The anti-Factor Xa activities (anti-Factor Xa activity) of native hFF aHSPG, and pure aHS and iHS and their specific anticoagulant activities have been studied as described in below. Table 2 below shows the anti-Factor Xa activity, AT content and prothrombin time (PT) of hFF. The prolonged PT seen in hFF was normalized when dilutions were done in plasma to complement Factor V and fibrinogen. AT was at the same level in hFF as in plasma. Comparison of anti-Factor Xa activity measured using dilutions of hFF in plasma or in buffer demonstrated that the elevated anti-Factor Xa activity could only be evidenced in the presence of normal AT concentration. It shows that hFF contains a potent anticoagulant activity with markedly prolonged PT, aPTT and TT. These prolonged times were not due to enhanced fibrinolysis, as D-dimer levels were not elevated. In keeping with previous observations, the levels in hFF of Factor V and fibrinogen were decreased in hFF as compared to their respective plasma concentrations. The reduced levels of Factor V and fibrinogen are insufficient to explain the profound prolongation in the aPTT and TT, which suggests the presence of an inhibitor of clotting. The TT measures clot formation by exogenously added thrombin and the extremely prolonged TT indicates the presence of a thrombin inhibitor, such as heparin/aHS. Indeed, hFF exhibits a high anti-Factor Xa activity, which indicates a strong heparin/aHS-like anticoagulant activity. This activity was lost when hFF was diluted in buffer, but retained with dilution in plasma, which suggests the anti-Factor Xa activity requires a plasma co-factor such as antithrombin.

TABLE 2

| | | | Anti- | Anti- | | |
|---|---|---|---|---|---|---|
| | | | Factor Xa | Factor Xa | | |
| | | | activity | activity | PT | PT |
| | | AT | (IU/ml) | (IU/ml) | (%) | (%) |
| | | (%) | diluted in | diluted in | diluted in | diluted in |
| Sample N^{o} | dilution | diluted in buffer | buffer | plasma | buffer | plasma |
| 3 | 1/1 | 107 | | >0.6 | 19 | |
| 3 | ½ | 47 | | | 13 | 78 |
| 3 | ¼ | 25 | 0.15 | 1.76 | 13 | 87 |
| 3 | ⅛ | 12 | <0.1 | 2.16 | 7 | 91 |
| 3 | 1/16 | | <0.1 | 2.4 | <5 | |
| mean ± sem | | | | 2.11 ± 0.23 | | |

The iHS fraction, which did not bind AT during the purification (see Example 1), exhibited undetectable anti-Factor Xa activity, even at the highest concentration tested of 200 μg/ml. (Table 3 below).

TABLE 3

| HS concen- | Anti-Factor Xa activity | | | | aHS | aHS |
|---|---|---|---|---|---|---|
| tration | aHS | aHS | iHS | iHS | aPTT | Thrombin |
| (μg/ml) | (IU/ml) | (IU/mg) | (IU/ml) | (IU/mg) | (sec) | time (sec) |
| 200 | | | <0.15 | nd | | |
| 3.13 | 0.51 | 163 | | | 64 | 36 |
| 2.50 | 0.35 | 140 | | | | |
| 1.56 | 0.33 | 211 | | | 47 | 26 |
| 1.25 | 0.19 | 152 | | | | |
| mean ± sem | | 167 ± 22 | nd | nd | | |

nd: not detectable;
aPTT reference time for plasma = 32 sec ± 6 sec;
TT reference value for plasma: 19 sec ± 5 sec.

In contrast, aHS had a very high anticoagulant activity (Table 3) that required extensive dilution of aHS to fall in the measuring range of the test and we obtained accurate results using 1-3 μg aHS/ml. The anticoagulant activity obtained for aHS was 167±22 IU/mg. This value is comparable to that of standard unfractionated heparin (133 IU/mg) and is the highest specific anticoagulant activity reported for HS. The aPTT and thrombin time were measured for aHS at concentrations of 1.5 and 3 μg/ml and showed prolonged times demonstrating the anticoagulant activity of hFF aHS. Taken together, these results demonstrate that hFF aHS has a potent anticoagulant activity mediated by AT, with a specific anti-Factor Xa activity similar to that of heparin.

### Anticoagulant Activity

The prothrombin time (PT) and the activated partial thromboplastin time (aPTT) measure the activation of the extrinsic and intrinsic pathway of the coagulation cascade, respectively. PT and aPTT are global coagulation assays, sensitive to decreased levels of coagulation factors. The thrombin time (TT) measures the inactivation rate of thrombin and the anti-Factor Xa activity specifically allows to quantify the inhibition of coagulation by heparin-activated AT, measuring heparin activity. aPTT and TT are prolonged in the presence of unfractionated heparin. Anticoagulant activity was assessed by hemostasis parameters analysis in native hFF and for aHS and iHS purified from hFF. Native hFF was supplemented with 1/10 volume of 0.13 M sodium citrate to prevent uncontrolled activation of coagulation as described above. Purified aHS and iHS were resuspended in 0.15 M NaCl. All measurements, except D-dimers, were made on Diagnostica Stago Analyzer (STA-R). Measurements were made using Automated aPTT from Bio-Merieux (Durham, N.C., USA) for the aPTT, human thrombin from Sigma (MO, USA) for TT, and STA Neoplastine CI 10 from Diagnostica Stago (Asniere, France) for PT; Diagnostica Stago STA deficient II, STA deficient V or STA deficient VII were used for Factor II, Factor V and Factor VII levels, respectively. Fibrinogen S (Diagonostica Stago) was used for Fibrinogen level, STACHROM AT III (Diagnostica Stago) for chromogenic AT assay. STA Rotachrom Heparin (Diagnostica Stago) was used for chromogenic heparin activity anti-Factor Xa assay with a standard curve done with unfractionated heparin. D-dimers were measured using a kit D-DI Test® (Diagnostica Stago). Assays were performed according to the manufacturer with, for some tests, the modifications described below. Dilutions in buffer were done using Owren-Koller buffer.

### Example 4

### Binding Ability of a Composition According to the Invention

The binding ability to P-selectin and L-selectin of the aHS and iHS is measured by surface plasmon resonance (SPR), the experiments were performed on a BIAcore 3000 according to the protocol described in Munoz et al., 2006, Biochem. Biophys. Res. Commun. 339:597-602. The binding constants for aHS and iHS with the selectins are shown in Table 4 below. P-selectin has a strong affinity with a low K_{D} of 0.89 nM for hFF aHS, with a fast Kₒₙ of 1.53×10⁴ M⁻¹sec⁻¹ and a very slow K_{off} of 1.4×10⁻⁵sec⁻¹, very distinct from the physiological ligand PSGL-1 (P-selectin glycoprotein ligand), that has a fast Kₒₙ and a fast K_{off}, allowing leukocyte rolling by rapid association-dissociation cycles. These data suggest that hFF contains aHS that are as potent as the highly sulfated porcine HS (std HS (Griffin et al., 1995, Carbohydr. Res. 276:183-197)) used as standard in this context. It is interesting to note that the binding of P-selectin to hFF aHS is 10 fold tighter than that of AT to heparin (K_{D} 8.5 nM).

TABLE 4

| | Kₒₙ | K_{off} | K_{D} |
|---|---|---|---|
| protein ligand | (M⁻¹sec⁻¹) | (sec⁻¹) | (nM) |
| | P-selectin | | |
| aHS | 1.53E+04 | 1.40E−05 | 0.89 |
| iHS | 3.03E+04 | 2.40E−04 | 7.9 |
| std HS | 1.61E+04 | 5.30E−06 | 0.33 |
| PSGL-1* | 4.40E+06 | 1.40E+00 | 320 |
| PSGL-1^{§} | 2.60E+04 | 1.20E−02 | 559 |
| | L-selectin | | |
| aHS | 2.60E+04 | 5.13E−04 | 20 |
| iHS | nd | nd | nd |
| std HS | 2.50E+04 | 9.20E−04 | 36.4 |
| | AT | | |
| heparin″ | 2.16E+07 | 3.63E−02 | 8.50 |
| bovine kidney HS^{#} | 1.70E+04 | 2.00E−03 | 118 |

Data for P-selectin binding to PSGL-1 (*) was from Mehta et al., 1998, J. Biol. Chem. 273:32506-32513, (^{§}) was from Simonis et al., 2007, Biochemistry. 46:6156-6164 and for AT binding to heparin (″) and to bovine kidney HS (^{#}) is from Munoz et al., 2006, above and Hernaiz et al., 2000, Biochem Biophys. Res. Commun. 276:292-297, respectively. The two sets of data for PSGL-1 were obtained with immobilised PSGL-1 and natural dimeric P-selectin (*), and with immobilised monomeric chimeric P-selectin-Fc, respectively.

The binding of L-selectin to hFF HS is very specific for aHS. L-selectin binds to aHS with slightly lower (2 to 10 fold) constants than P-selectin but L-selectin does not bind at all to iHS, while P-selectin binds to iHS about 10 fold less tightly than to aHS. Thus, the binding of L-selectin to hFF aHS is very specific indicating that the binding sequence to AT and to L-selectin are linked on the same HS chains. HS have been shown to play a major role in L-selectin-mediated monocyte attachment to activated endothelial cells (Giuffre at el., 1997, J. Cell Biol. 136:945-956). Non-anticoagulant HS efficiently inhibit selectin-mediated cell adhesion and prevent acute inflammation (Xie et al., 2000, J. Biol. Chem. 275:34818-34825). These data suggest that hFF aHS could exert anti-inflammatory activity by inhibition of P- and L-selectin binding to ligands such as PSGL-1 and limit leukocyte infiltration in the context of inflammation.

1. A method for the preparation of a glycosaminoglycan composition, comprising the steps of:
(a) Providing a follicular fluid sample;
(b) Purifying the said follicular fluid sample by ion exchange chromatography to eliminate the bulk of the proteins and recover the charged proteoglycans and glycoaminoglycans;
(c) Digesting the purifed proteoglycans and glycoaminoglycans obtained under step (b) with chondroitinase ABC to eliminate non-heparan sulfate species;
(d) Isolating of free glycoaminoglycans from high molecular weight proteoglycans by gel filtration;
(e) Recovering heparan sulphate glycoaminoglycan chains by β-eliminative cleavage on the isolated high molecular weight proteoglycans obtained under step (d);
(f) Removing proteins from the recovered heparan sulphate glycoaminoglycan chains fraction obtained under step (e) by phenol extraction;
(g) Recovering a glycosaminoglycan composition from the protein free fraction obtained under step (f) by ethanol precipitation.

2. A method according to claim 1 wherein the method further comprises the following steps after step (g):
(h) Isolating the anticoagulant (aHS) and non-anticoagulant (iHS) heparan sulphate fractions from the composition obtained under step (g) by antithrombin affinity gel chromatography.
(i) Recovering separately the two fractions obtained under step (h).

3. An isolated glycosaminoglycan composition obtainable by a method according to claim 1.

4. An isolated glycosaminoglycan composition comprising: (a) at least about 2% 3-O-sulfated glucosamines (GlcNS3S or GlcNS3S6S); and (b) about 0.5 to 2 sulfate per disaccharide unit.

5. A composition according to claim 4, further comprising at least about 20% of non-sulfated disaccharides and at least about 60% sulfated disaccharides.

6. A composition according to claim 4, further comprising at least about 10% 2-O-sulfated uronic acids (IdoA2S) and at least 70% non-sulfated uronic acids.

7. A composition according to claim 4, further comprising at least about 15% 6-O-sulfated glucosamines (GlcNac6S, GlcN6S or GlcNS3S6S).

8. A composition according to claim 4, wherein the composition contains at least about 10% glycosaminoglycans, with an anti-Factor Xa specific activity of at least 50 UI/mg.

9. A composition according to claim 4, wherein the composition contains glycosaminoglycans having a chain size of at least about 10 kDa and wherein those chains contain at least two 3-O-sulfated glucosamines.

10. A composition according to claim 4 comprising:
(a) 2 to 30% of 3-O-sulfated glucosamines;
(b) 0.5 to 2 sulfate per disaccharide unit;
(c) 20 to 40% of non-sulfated disaccharides and 60 to 80% of sulfated disaccharides;
(d) 10 to 30% of 2-O-sulfated uronic acids and 70 to 90% non-sulfated uronic acids;
(e) 15 to 50% of 6-O-sulfated glucosamines;
(f) at least about 10% of glycosaminoglycans with an anti-Factor Xa specific activity of at least 50 UI/mg; wherein the composition contains glycosaminoglycans having a chain size of 10 to 50 kDa and wherein those chains contain two to fifteen 3-O-sulfated glucosamines.

11. A pharmaceutical preparation comprising at least one glycosaminoglycan composition according to any one of claims 3 or 4 and pharmaceutically acceptable carrier or excipient.

12. A kit comprising at least one glycosaminoglycan composition according to any one of claims 3 or 4.

13. Use of a glycosaminoglycan composition according to any one of claims 3 or 4 for the preparation of a medicament for the prevention and/or treatment of thrombotic diseases, cell proliferation disorders, proteolysis and inflammation mediated cell invasion and infertility.

14. A glycosaminoglycan composition according to any one of claims 3 or 4 for preventing or treating thrombotic diseases, cell proliferation disorders, proteolysis and inflammation mediated cell invasion and infertility.

15. A method for preventing and/or treating a disease comprising the administration of a therapeutically effective amount of a glycosaminoglycan composition or a pharmaceutical composition thereof according to any one of claims 3 or 4 in a mammal in need thereof and wherein the disease is selected from thrombotic diseases, cell proliferation disorders, proteolysis and inflammation mediated cell invasion and infertility.

16. A method of monitoring ovulation comprising the step of:
(a) Providing a blood sample from a female patient;
(b) Measuring the amount of anticoagulant heparan sulfate (aHS) in the sample provided under step (a);
(c) Comparing the amount of aHS measured under step (b) with an amount of aHS standard for a female in a non-ovulatory period.

17. A kit for monitoring ovulation, the kit comprising:
(a) at least one blood sample testing device that provides a readable signal proportional to the aHS concentration in a blood sample;
(b) an electronic monitor having reading means to read the readable signal obtained under step (a) and incorporating computer means to interpret the readable signals and to determine therefrom in conjunction with data from previous blood sample tests whether the event of ovulation in the current cycle has just occurred.

18. A device comprising a kit according to claim 17.
